(19) United States
(12) Patent Application Publication
(10) Pub. No.: US 2024/0071575 A1
(43) Pub. Date: Feb. 29, 2024

(54) USING COMPUTER SIMULATION FOR RANKING MATERIALS FOR POST COMBUSTION CARBON CAPTURE

(71) Applicants: INTERNATIONAL BUSINESS MACHINES CORPORATION, Armonk, NY (US); EXXONMOBIL TECHNOLOGY AND ENGINEERING COMPANY, ANNANDALE, NJ (US)

(72) Inventors: Rodrigo Neumann Barros Ferreira, Rio De Janeiro (BR); Ricardo Luis Ohta, SAO PAULO (BR); Yogesh V. Joshi, Bridgewater, NJ (US); Mathias B. Steiner, RIO DE JANEIRO (BR); Tonia Elengikal, Yorktown Heights, NY (US); Binquan Luan, Yorktown Heights, NY (US); Ashish B. Mhadeshwar, Garnet Valley, PA (US); Jayashree Kalyanaraman, Clinton, NJ (US); Anantha Sundaram, Annandale, NJ (US); Sumathy Raman, Annandale, NJ (US)

(21) Appl. No.: 17/899,271

(22) Filed: Aug. 30, 2022

(51) Int. Cl. G16C 20/30

(52) U.S. Cl. CPC G16C 20/30

(57) ABSTRACT

Ranking materials for post combustion carbon capture by characterizing sorbent materials with a molecular model workflow that generates microscopic figures of merit for materials by microscopic properties; and evaluating the materials from the molecular model workflow with a process model workflow that generates macroscopic figures of merit for process steps of a carbon recovery process. The materials for applicability as a sorbent material are ranked using a combined microscopic performance and macroscopic process feasibility generator that ranks the materials according to the microscopic figures of merit for materials and the macroscopic figures of merit for the process steps.

### BACKGROUND

The present invention generally relates to computer simulation of carbon capture, and more particularly to quantifying CO₂ capture materials performance at given pressure and temperature (P,T) conditions using computer simulation.

Carbon capture and storage (CCS) or carbon capture and sequestration is the process of capturing carbon dioxide (CO₂) before it enters the atmosphere, transporting it, and storing it (carbon sequestration) for centuries or millennia. Usually the CO₂ is captured from large point sources, such as a chemical plant or biomass power plant, and then stored in an underground geological formation. The aim is to prevent the release of CO₂ from heavy industry with the intent of mitigating the effects of climate change. CO₂ can be captured directly from an industrial source, such as a cement kiln, using a variety of technologies; including absorption, adsorption, chemical looping, membrane gas separation or gas hydration. It has been determined that for accelerating the discovery of optimized materials for carbon capture, there is a need to computationally quantify the carbon dioxide (CO₂) capture potential of sorbent materials at given pressure and temperature.

### SUMMARY

In accordance with an embodiment of the present invention, a computer-implemented method is provided for ranking materials for post combustion carbon capture. The computer-implemented method may include characterizing sorbent materials with a molecular model workflow that generates microscopic figures of merit for materials by microscopic properties; evaluating the materials from the molecular model workflow with a process model workflow that generates macroscopic figures of merit for process steps of a carbon recovery process such as a carbon capture process or carbon separation process; and ranking the materials for applicability as a sorbent material using a combined microscopic performance and macroscopic process feasibility generator that ranks the materials according to the microscopic figures of merit for materials and the macroscopic figures of merit for the process steps.

In another embodiment, a system for ranking materials is provided for post combustion caron capture s provided that includes a hardware processor; and a memory that stores a computer program product. The computer program product when executed by the hardware processor, causes the hardware processor to characterize sorbent materials with a molecular model workflow that generates microscopic figures of merit for materials by microscopic properties; and evaluate the materials from the molecular model workflow with a process model workflow that generates macroscopic figures of merit for process steps of a carbon recovery process. The computer program product of the system may further rank the materials for applicability as a sorbent material using a combined microscopic performance and macroscopic process feasibility generator that ranks the materials according to the microscopic figures of merit for materials and the macroscopic figures of merit for the process steps.

In yet another embodiment, a computer program product is provided for ranking materials for post combustion carbon capture. The computer program product may include a computer readable storage medium. The computer readable storage medium may have computer readable program code embodied therewith, the program instructions executable by a processor to cause the processor to characterize, using the processor, sorbent materials with a molecular model workflow that generates microscopic figures of merit for materials by microscopic properties; and evaluate, using the processor, the materials from the molecular model workflow with a process model workflow that generates macroscopic figures of merit for process steps of a carbon recovery process. The computer readable storage medium also includes instructions that can rank, using the processor, the materials for applicability as a sorbent material using a combined microscopic performance and macroscopic process feasibility generator that ranks the materials according to the microscopic figures of merit for materials and the macroscopic figures of merit for the process steps.

In yet another aspect, a method for a separation process implementation is described. In one embodiment, the method includes characterizing sorbent materials with a molecular model workflow that generates microscopic figures of merit for materials by microscopic properties; and evaluating the materials from the molecular model workflow with a process model workflow that generates macroscopic figures of merit for separation process steps of interest. The method can further rank the materials for applicability as a sorbent material using a combined microscopic performance and macroscopic process feasibility generator that ranks the materials according to the microscopic figures of merit for materials and the macroscopic figures of merit for the process steps; and select highest ranked materials from the ranking for integration as sorbent materials into at least one of a desorber and an adsorber of a separation process employing at least one of a pressure swing adsorption cycle and a temperature swing adsorption cycle. The method can further include performing separation using the at least one of the desorber and adsorber with the sorbent materials. The separation process can be selected from carbon recovery, carbon capture, air separation, natural gas separation, hydrogen purification, ammonia separation, N₂ purification, O₂ purification, H₂O removal, bio gas separation and combinations thereof.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 illustrates one embodiment of an exemplary environment for the systems and methods ranking materials for post combustion carbon capture, in accordance with one embodiment of the present disclosure.

FIG. 2 is a flow chart/block diagram illustrating a system for post combustion carbon capture, in accordance with one embodiment of the present disclosure.

FIG. 3 illustrates one embodiment of a flow chart for a molecular level screening workflow, in accordance with one embodiment of the present disclosure.

FIG. 4 is an illustration of the largest sphere along a free path (D_{isfs}), largest free sphere (D_{fs}) and largest included sphere (Dᵢₛ) geometric figures-of-merit overlaid to a crystal structure.

FIG. 5 is a flow chart/block diagram illustrating one embodiment of a flow chart for a process modeling workflow, in accordance with one embodiment of the present disclosure.

FIG. 6 is a side cross-sectional view of an adsorption bed model for dynamic model formulation, in accordance with one embodiment of the present disclosure.

FIG. 7 is a schematic representation of the steps of PSA/TSA cycle including adsorption, purge, desorption and cooling.

FIG. 8 is a block diagram illustrating a system that can incorporate the system for post combustion carbon capture, that are depicted in FIG. 2, in accordance with one embodiment of the present disclosure.

FIG. 9 depicts a computing environment according to an embodiment of the present disclosure.

### DETAILED DESCRIPTION

The methods, systems and computer program products described herein are directed to ranking candidate solid sorbent materials for CO₂ post combustion capture based on their microscopic structures. In the methods and structures of the present disclosure a molecular modeling workflow is coupled with a process modeling workflow to derive macroscopic material performance and figures of merit. In some embodiments, the objective of systems, methods and computer program products is to predict CO₂ separation figures of merit to guide lab scale development and synthesis of post combustion CO₂ solid sorbents.

A “sorbent” is a material used to absorb or adsorb liquids or gases. In the present case, the sorbents are for CO₂ gas. Solid sorbents include a diverse range of nanoporous solids, such as zeolites, metal organic frameworks (MOF), zeolitic imidazolate frameworks (ZIF) and porous polymer networks (PPN) are all examples of sorbent materials. Solid sorbents can selectively absorb carbon dioxide (CO₂) without bond formation, i.e., via physisorption, which drastically lowers the regeneration energy and, therefore, the operating cost.

Metal organic frameworks have a customizable porous structure. They are composed of two types of building blocks, i.e., organic linkers and metal ions, which can be combined to form nano porous structures with channels whose diameters can range from a few to tens of Angstroms. The chemical and geometrical aspects can be leveraged tuned independently to optimize both the absolute (adsorption capacity) and relative (adsorption selectivity) metrics that determine what is a good carbon CO₂ capture material.

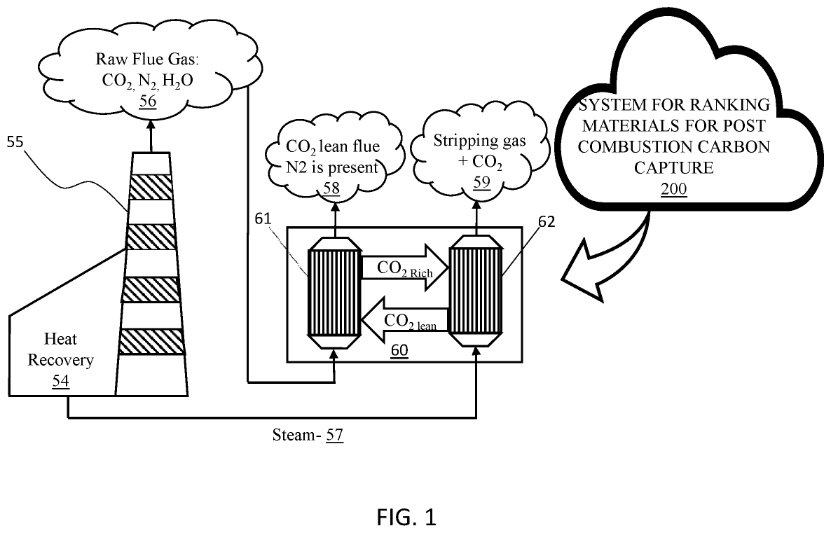

The methods, systems and computer program products are now described in greater detail with reference to FIGS. 1-9.

FIG. 1 illustrates one embodiment of an exemplary environment for the systems and methods ranking materials for post combustion carbon capture. Illustrated is a natural gas power plant 55 that is being treated with a pressure and/or temperature swing adsorption 60 using a adsorption column 61 and a desorption column 62. The adsorption column 61 and the desorption column 62 include sorbent materials, such as solid sorbent materials, e.g., such as zeolites, metal organic frameworks (MOF), zeolitic imidazolate frameworks (ZIF) and porous polymer networks (PPN) are all examples of sorbent materials. In the example depicted in FIG. 1, the natural gas power plant 55 produces flu gases of carbon dioxide (CO₂), nitrogen (N₂) and water (H₂O). Pressure swing adsorption (PSA) cycle and Temperature swing adsorption (TSA) cycles gas pressure and/or gas temperature around an adsorbent media (sorbent materials) to selectively adsorb certain components of a gas, allowing others to be selectively discarded. In some embodiments, an efficient carbon capture process separates CO₂ and N₂, achieves high purity CO₂, and lower energy use (Steam -57). The adsorption column 61 selectively absorbs CO₂ from the flu gasses. In the adsorber 61, the regenerated adsorbent material selectively adsorbs CO₂ that is present in the flue gas stream 56. Typically, CO₂ separation purity and recovery of around 90% are targeted in order to achieve a flue gas stream 58 at the exit of the adsorber 61 with a significantly reduced CO₂ concentration. Since adsorption is generally an exothermic process, heat is released in the adsorber 61 and therefore active cooling of the adsorbent material may be required to maintain the desired operating temperature in the adsorber. The loaded adsorbent material is continuously transported into the desorber 62 or the adsorption column itself is switched to desorption mode by using valves where it is heated up to a higher temperature. This temperature shift decreases the CO₂ capacity of the adsorbent material so that CO₂ gets desorbed and the adsorbent material regenerated again. The temperature increase of the adsorbent material and the fact that desorption of CO₂ is an endothermic process requires a constant heat supply to the desorber 62. Apart from the temperature increase in the desorber 62, the desorption of CO₂ is further driven by the utilization of steam 57 as stripping agent. The supplied steam 57 decreases the CO₂ partial pressure in the desorber 62 and thereby helps to achieve deep regeneration of the adsorbent material. Ideally, the off-gas 59 of the desorber 62 only contains CO₂ and steam so that after condensation, CO₂ can be obtained at a high purity. The supplied steam 57 may be captured at the natural gas power plant 55. The system for ranking materials for post combustion carbon capture 200 is depicted in FIG. 1 as a cloud based service that aids in the selection of sorbent materials, such as those used in the adsorber 61 and desorber 62 of the PSA/TSA process 60. It is noted that the natural gas power plant 55 and the pressure swing adsorption (PSA) cycle and/or temperature swing adsorption (TSA) cycles capture process are provided for illustrative purposes only. Other carbon generating environments and methods of capture using sorbent materials are equally applicable for use with the systems for ranking sorbent materials. Similarly other separations including but not limited to air separation, natural gas separation, bio gas separations, hydrogen purification, ammonia separation, N₂ purification, O₂ purification, H₂O removal can also be assessed using systems for ranking sorbent materials.

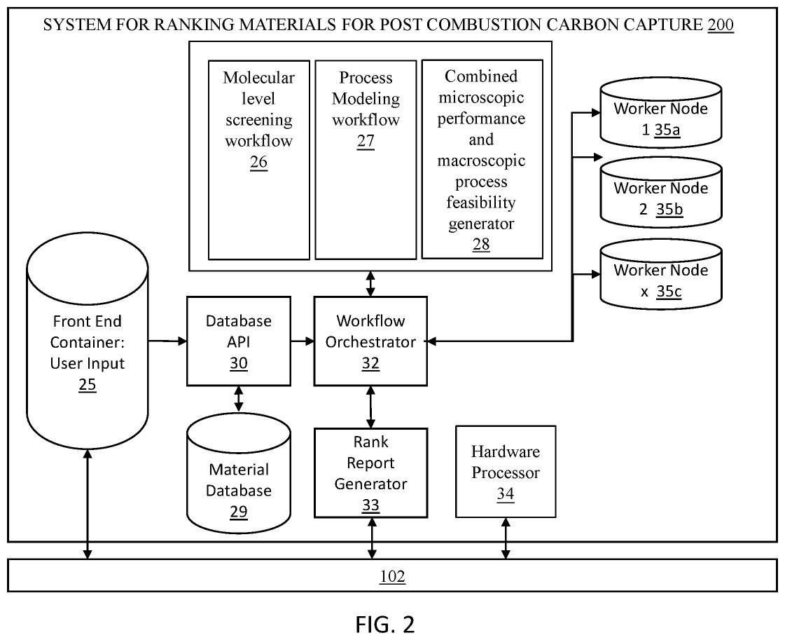

FIG. 2 illustrated one embodiment of a system for ranking materials for post combustion carbon capture 200 that includes a molecular modeling workflow 26, a process modeling workflow 27, and a combined microscopic performance and macroscopic process feasibility generator 28.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Referring to FIG. 2, the molecular modeling workflow 26 may include microscopic figures of merit. These microscopic figures of merit may include characterizations of a material, such as loading, heat capacity, and heat transfer, as well as other material characterizations useful in determining suitability of a material for separation applications.

In one example, the microscopic figures of merit include data from an adsorption isotherm for a particular solid sorbent material. Adsorption isotherms can used to describe the quantity of adsorbate on the surface as a function of its pressure at constant temperature. By measuring adsorption and desorption performance from the data provided by an adsorption isotherm for a particular material the swing capacity for the material can be characterized.

For example, the process modeling workflow 27 may include macroscopic figures of merit. This can include performance characterizations of a material from a process perspective, e.g., recovery, purity, productivity, energy use. These processes are the process steps of a carbon recapture process. An example of which is illustrated by the environment depicted in FIG. 1. Recovery can be characterization of the gasses 58 emitted by the adsorber 61, which are carbon lean and N₂ rich. Purity can be a characterization of the gasses 59 emitted by the desorber 62, which should be CO₂ pure excluding condensable gases such as steam. Energy use is a characterization of the steam 57 used at the desorber 62. Productivity is a characteristic of the stream 59 emitted by the desorber 62 and inversely dependent on total volume of the adsorber 61 and the desorber 62. All of these characterizations are specific to a material being used in a particular process, or sub-process.

The combined microscopic performance and macroscopic process feasibility generator 29 employs the process modeling frameworks to reduce the number of possible material compositions that are suitable for sorbent applications from being on the order of 10,000 materials to a suitable set of materials that can be on order of 10's of materials. For example, the molecular modeling workflow 27 can generate on the order of 10,000 material. A simple model for process based screenings, such as an independent use of the macroscopic figure of merit from the process modeling workflow 27, can reduce that number to 100's of materials. However, by combining the figures of merit for the molecular modeling workflow 26 and the process modeling workflow 27, the combined microscopic performance and macroscopic process feasibility generator 28 can reduce the number of suitable material compositions and characterizations to the order of 10's of materials, which is advantageous for considering sorbent materials, such as metal organic frameworks. The objective is to predict gas separation figures-of-merit to guide development and synthesis of solid sorbents, including but not limited to the applications in the separation of CO₂/N₂, CO₂/CH₄, O₂/N₂, alkenes/alkanes, Xe/Kr, Xe/Ar, among others.

In some embodiments, the combined microscopic performance and macroscopic process feasibility generator 29 comprises of a multi-step and multi-criteria optimizer that orders the materials using combined trade-off metrics considering different dimensions of performance enhancement.

The recommended sorbent materials can be integrated into the adsorber 61 and/or desorber 62 of a pressure and/or temperature swing adsorption (PSA/TSA) process 60 depicted in FIG. 1 during which carbon capture can be performed. Ranked materials can automatically be implemented in the order suggested by the combined microscopic performance and macroscopic process feasibility generator 28 until an optimized systems is realized.

Referring to FIG. 2, the system for ranking materials for post combustion carbon capture 200 that includes the molecular modeling workflow 26, the process modeling workflow 27, and the combined microscopic performance and macroscopic process feasibility generator 28 may be integrated into an architecture in which a user interacts with the application through a Front-End Container 25 that provides the user interface. The Front-End Container 25 uses the Workflow Orchestrator 32 to run multi-stage simulations on a cloud-computing Cluster with multiple Worker Nodes 35a, 35b, 35c. The simulations employ the molecular level screening workflow 26, the process modeling workflow 27 and the combined microscopic performance and macroscopic process feasibility generator 28. The material properties resulting from these simulations are stored in a Database 31 hosted on the Cloud.

The architecture may include an application programming interface (API)(database API 30) that provides web endpoints to modify and query the material database 29 from the front end container that provides the user input 25. In some embodiments, the database API 30 is a REST API container provides web endpoints to modify and query the database 29 from the front-end container 25. A “REST API” (also known as RESTful API) is an application programming interface (API or web API) that conforms to the constraints of REST architectural style and allows for interaction with RESTful web services. REST stands for representational state transfer.

The system 200 depicted in FIG. 2 may be deployed as a Platform-as-a-Service (PaaS) model and deployed as containerized applications in a cloud-based cluster. The architecture of the system 300 can be summarized as follows: a NoSQL database (material database 29) that stores material definitions, a REST API (database API 30) that provides access to the database contents, a workflow scheduler (workflow orchestrator 32) that allows for submitting screening workflows for each material and a interface (front end containing: user input 25) that facilitates user interaction with all other components.

The NoSQL database (material database 29) stores nano-porous materials definitions at an atomistic level as encoded by the Crystallographic Information File (CIF) format, alongside metadata (e.g., name, class, source, presence of disorder, and presence of an open metal site). The database REST API allows for creating, reading, updating, and deleting material entries and their physical properties. The workflow scheduler 32 allows for submitting multi-step screening workflows that can fully characterize a material from the atomistic point of view. The interface (front end container 25) takes as input the list of materials to be screened, the adsorbate gas composition and the conditions—temperatures and pressures—under which the molecular adsorption simulations should performed. All applications run on a cloud-based cluster.

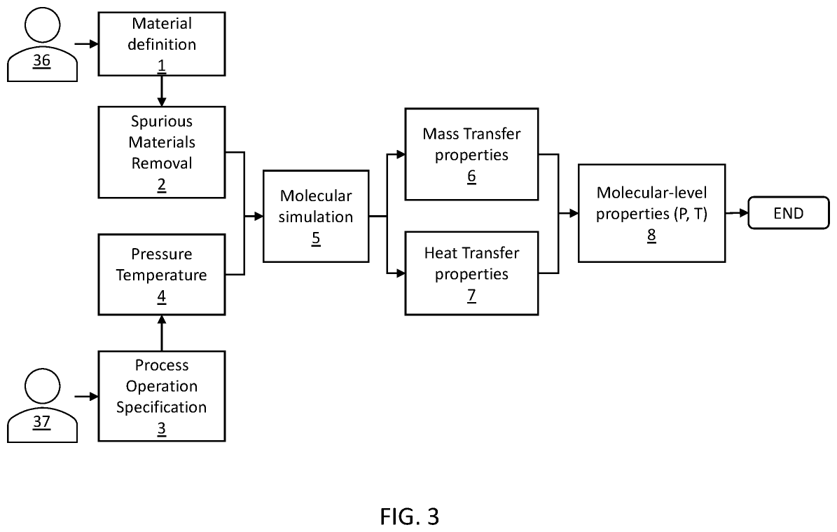

FIG. 3 illustrates one embodiment of a flow chart for a molecular level screening workflow. The molecular screening workflow depicted in FIG. 3 may be run by the molecular level screening workflow 26 of the system depicted in FIG. 2. As noted, the molecular modeling workflow is could to processing modeling workflow to derive macroscopic performance/figures of merit. The objective of coupling the molecular modeling workflow with the molecular level screening workflow is to predict gas separation figures-of-merit to guide lab-scale development and synthesis of solid sorbents, including but not limited to the applications in the separation of CO₂/N₂, CO2/CH₄,O₂/N₂, alkenes/alkanes, Xe/Kr, Xe/Ar, among others.

Referring to FIG. 3, in some embodiments, the molecular-level screening workflow starts by a user 36 querying the database REST API (database API 30) for candidate materials to be screened at block 1. At block 1 of FIG. 3, the component responsible for performing the molecular modeling may include a material definition that serves as input to molecular simulation engine at block 5. The definitions can include a list of atom species in the compositions of interest, as well as crystallography data, such as crystal structure, etc. The selection may be random or focus on a particular subset of materials, such as those from a particular source or class. For example, the material selection may be a solid sorbent, which can include a diverse range of nanoporous solids, such as zeolites, metal organic frameworks, zeolitic imidazolate frameworks and porous polymer networks. The database query returns the location in the cloud-based cluster of the corresponding CIF files of each one of the materials in the selected subset. The database query may employ the material database 29 from FIG. 2.

Referring to FIG. 3, the user identified by reference number 36 may be responsible for providing a (set of) material definitions, such as Crystallographic Information Files (CIF), as input to the simulation engines at block 5, and the user identified by reference number 37 may be responsibility for providing the range of pressures and temperatures of interest based on separation process conditions. The user identified by reference number 36 enters material definitions, while the user identified by reference number 37 enters process conditions relevant for the process at which the material would be operating in an adsorption, desorption setting at block 4. Block 3 includes the user 37 entering process operations specifications. The process operations specifications can include an operation, such as adsorption and/or desorption. Although FIG. illustrates two users 36, 37, the same party can before both functions.

In some embodiments, in order to keep the structure definition chemically accurate, spurious structures are removed at block 2 based on the specific microscopic features, such as coordination number, and charge ranges expected for similar materials class. By “spurious” it is meant that there can be elements in the microstructure that are not intended to be included For example, unbound solvent molecules could be removed from the CIF. For example, elements that are far away from the known features of a chemical microstructure may be considered unbound, and removed as being spurious. Known algorithms can be used to calculate spatial relationships, and if an element exceeds a set threshold, it can be designated as being spurious, and removed. Microscopic features can be extracted directly from the CIF (charges).

Turning to block 5, the molecular transport properties are calculated at relevant temperature and pressure for the separation process specified at block 3 to maximize the accuracy of these properties. The molecular simulation engine at block 5 is responsible for computing molecular transport properties at block 6, such as diffusivity, loading and mass transfer. The molecular simulation engine at block 5 can also compute heat transfer properties at block 7, such as heat capacity and heat conductivity.

The molecular ensembles under which these simulations are performed may change in accordance with the type of desired molecular-level property. For instance, the molecular diffusivity coefficient may be calculated at infinite dilution (P→0) with N=1 gas molecule at the desired temperature T, under the Canonical (NVT) ensemble. Alternatively, the equilibrium molecular loading is best calculated under a constant chemical potential in the Grand Canonical (μLVT) ensemble.

In some embodiments, going back to block 5, the molecular simulation can be a sequence of computations and simulations that take the CIF files as direct input. The molecular modeling framework employs a suite of containerized scientific applications to compute geometric and molecular adsorption figures-of-merit, which are later appended to their respective material entry in the database 29 to form a consolidated record of material candidates.

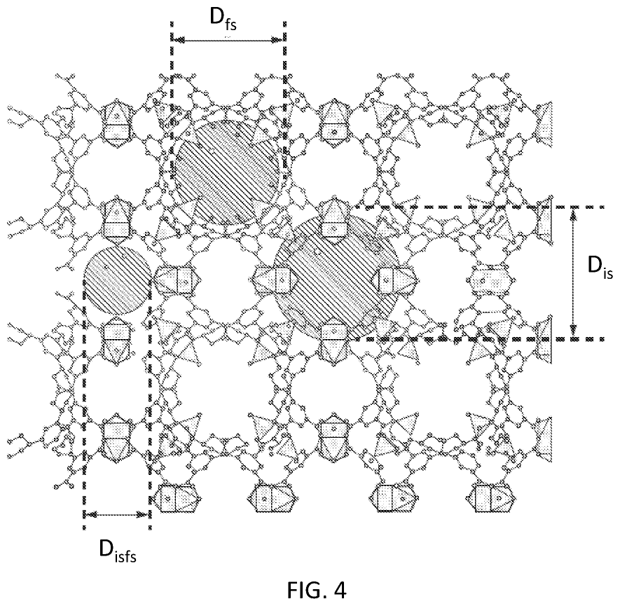

The first block in the sequence, is the creation of an asymmetric unit cell with P1 symmetry, in which all atoms are explicitly listed in the CIF file, instead of relying on symmetry operations to build the full crystal cell. The asymmetric unit cell is provided as input to software for performing high-throughput geometry-based analysis of porous materials and their voids. The main code provides capabilities to calculate parameters describing pore sizes are the diameters describing: (1) the largest included sphere (Dᵢₛ), (2) the largest free sphere (D_{fs}), and (3) the largest included sphere along the free sphere path (D_{isfs}). Illustration of the D_{isfs}, D_{fs} and Dᵢₛ geometric figures-of-merit overlaid to the crystal structure is depicted in FIG. 4.

Several geometric figures-of-merit are calculated using a 1.4 Å probe radius: gravimetric and volumetric (non-)accessible surface area, gravimetric (non-)accessible volume, (non-)accessible volume fraction, density, unit cell volume, number of non-accessible pores (pockets), diameter of largest free sphere, diameter of largest included sphere and diameter of largest included sphere along free path. These geometric figures-of-merit, such as the ones shown in FIG. 4, are stored in the database automatically, via a call to the database REST API (database API 30) which updates the record of each material to include the geometric information.

The CIF file containing the asymmetric unit cell is then provided as input to the calculation of partial atomic charges under an EQeq scheme. The extended charge equilibration (EQeq) scheme computes atomic partial charges using the experimentally measured ionization potentials and electron affinities of atoms. The result of this calculation is a new CIF file with partial atomic charges assigned to each atom. The new charge-aware CIF file is used as input to RASPA software for calculating an energy grid for CO₂, N₂ and H₂O molecules. RASPA is a software package for simulating adsorption and diffusion of molecules in flexible nanoporous materials. The code implements the latest state-of-the-art algorithms for molecular dynamics and Monte Carlo in various ensembles.

In some embodiments, a 12.8 Å cutoff radius is used for the long-range interaction and the energy is computed on a 0.1 Å grid with 10⁻⁶ relative precision. In some embodiments, the grid is calculated in a supercell with perpendicular lengths that are, at least, twice as large as the cutoff radius.

In some embodiments, the CO₂ and N₂ molecules are represented by their respective Transferable Potentials for Phase Equilibria (TraPPE) models, while the H₂O molecule is represented by a TIP5P model enhanced for use with Ewald sums. The Transferable Potentials for Phase Equilibria (TraPPE) family of force fields is a collection of functional forms and interaction parameters useful for modeling complex chemical systems with molecular mechanics simulation techniques. TIP5P is a five-site rigid model of water. The molecular simulation at blocks, 5, 6 and 7 use a hybrid UFF/Dreiding Lennard-Jones parameter set with Lorentz-Berthelot mixing rules to model the framework-molecule interactions.

Grand Canonical Monte Carlo (GCMC) simulation is performed using the pre-computed energy grid and the charge-aware CIF file as inputs, encompassing 10,000 cycles for each temperature and pressure values. The resulting timeseries for the number of adsorbate molecules in the simulation box as a function of Monte Carlo cycles is subject to an equilibration routine using the Marginal Standard Error Rule (MSER) as implemented by the pyMSER package. For each simulation, at a given temperature and pressure, the time series is processed and an equilibrated average loading value is calculated alongside a representative uncertainty metric. The uncorrelated standard deviation is the representative uncertainty metric, which is calculated by taking the equilibrated part of the timeseries, as provided by the MSER method, and splitting it into chunks of length tcorr, where tcorr represents the autocorrelation time. The average of each (uncorrelated) chunk is then taken and the standard deviation is calculated for the collection of chunk averages. Single-component gas adsorption isotherms are simulated for CO₂, N₂ and H₂O at temperature and pressure at block 8. For example, the temperature (T) may be set 298 K, 323 K, 348 K and 373 K, and the pressure may be set at P=0.0001 to 1 bar for H₂O and from P=0.01 to 50 bar for CO2 and N₂. The combination of all these molecular-level properties at given pressure and temperature is the outcome at block 8. Each isotherm curve, for each adsorbate gas, is later stored in the materials database 29 alongside the geometric properties for later analysis.

Following the molecular level screening workflow, the process modeling workflow is performed for providing a combined microscopic performance and macroscopic process feasibility measurement, e.g., the ranking of materials for post combustion carbon capture.

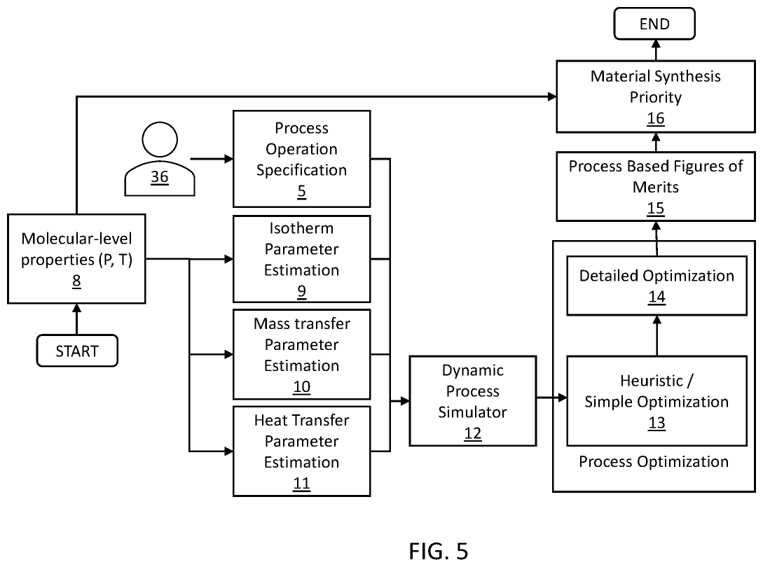

FIG. 5 illustrates one embodiment of a flow chart for a process modeling workflow 27 illustrated in FIG. 2. The first step of process modeling framework 27 includes building functional form of mass and heat transport properties as function of temperature, pressure and composition to be used in process models, as illustrated by blocks 9, 10 and 11.

The process operation is specified as initial set of operating conditions at block 5, such as adsorption, desorption temperature, cycle time for adsorption, purge, desorption, cooling steps and flow rates during individual cycle steps. These elements can be entered by the user 36, and are consistent with block 3 of the molecular modeling framework that is described above with reference to FIG. 2.

The cyclic pressure and/or temperature swing adsorption (PSA/TSA) process 60, as illustrated in FIG. 1, includes adsorption, purge, desorption, and cooling steps, which can be simulated using a dynamic process simulator until cyclic steady state is reached. The dynamic process simulation at block 12 can be built using a process modeling software such as gPROMS®, Aspen®, ProSim DAC®, MINSA, etc. In these examples of process modeling, mass, momentum and energy balance equations are solved in spatio-temporal domain to generate concentration and temperature profiles. The adsorption column model (for the absorber 61 depicted in FIG. 1) is subjected to steps in PSA/TSA cycle and output is used to evaluate both optimal cycle parameters for simple optimization at block 13, as well as detailed optimization at block 14. The detailed optimization at block 14 can use variety of optimization algorithms (Simplex, GA etc) which uses the formulated process model to calculate process figures of merits at block 15 to be used in the multi-objective fitness function.

The dynamic process is used in both heuristic (simple) and detailed optimizer to optimize initial process parameters such as cycle times, adsorption/desorption temperatures and flow rates for each of the materials under consideration. The cycle times (adsorption time, purge time, desorption time, cooling time and desorption temperature) are determined heuristically to run simple process optimization for each material at block 13. These variables, adsorption temperature, additional flow rates are further optimized using detailed process model optimization block at block 14.

The objective of the optimization at blocks 13 and 15 is to maximize productivity, minimize energy consumption while achieving CO₂ purity specification required from the process for every material.

The process based figures of merits are evaluated using output from dynamic process model at block 15. Turning to block 16, based on the molecular-based figures of merit (from block 8, as determined using the method in FIG. 3), and the process-based figures of merits, the synthesis priority order of the materials is determined for certain pareto-optimal criteria, such as minimizing energy consumption, maximizing selectivity, maximize productivity or combination of multiple criteria.

The combination of the molecular modeling framework 26, as illustrated by the method depicted in FIG. 3, with process based figures of merit provided by the process modeling workflow, as illustrated by the method described with reference to FIG. 5, can provide process-level figures of merit based on microscopic properties of material.

In some examples, the isotherm model at block 9 and the parameter estimations at block 10 and 11 can employ temperature dependent single, dual and triple site Langmuir models for fitting the isotherm data. The isotherm models are described by following equations (Eq.) 1 and 2. In these equations, ΔH_{k,i} and ΔS_{k,i} are enthalpy and entropy of adsorption for species k and site i. q_{sat,i} is saturation capacity of site i. The isotherm parameters are estimated by minimizing sum square of residuals between simulated loading and predicted loading using isotherm models, using L-BFGS-B algorithm implemented in SciPy optimize package. Out of three isotherm models, the isotherm model with minimum sum square error is selected for process modeling. Equations (Eq.) 1 and 2 are as follows:

$${q}_{eq , k} = {\sum }_{i = 1}^{3} \frac{{q}_{sat , i} {b}_{k , i} {p}_{k}}{1 + {b}_{k , i} {p}_{k}} \qquad ( 1 )$$

$${b}_{k , i} = {e}^{- ( Δ {H}_{k , i} - T Δ {S}_{k , i} ) / RT} \qquad ( 2 )$$

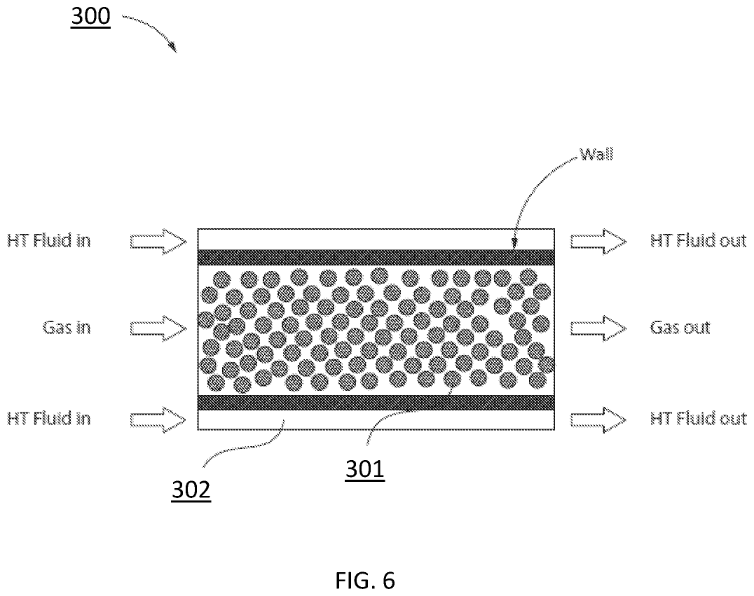

Dynamic process-model formulation and implementation at blocks 12, 13 and 14 of the method illustrated in FIG. 5 may includes a rigorous process model to do Temperature Swing Adsorption (TSA) in fixed bed configuration is developed and built in gPROMS® model builder software. As shown in FIG. 6, a pack bed model 300 is modeled as one dimensional bed of sorbent material 301 with heat and mass transfer along the axial dimension. Thermal management within the bed is achieved the fluid 302 surrounding the bed, which could either be considered as the ambient atmosphere or a temperature controlled fluid depending upon the scale of the unit. The basic assumptions used while formulating the model description include that competitive sorption of CO₂, N₂ and H₂O is modeled by combining their respective pure component isotherms of into a multi-component Langmuir isotherm. Variation in concentration, temperature and momentum across the radial dimension of the bed as well as within the internal tubes are assumed to be negligible. Mass transfer resistance is modeled using linear driving force based on the lumped macroscopic and microscopic diffusional resistance.

The mass balances of gas and the solid phases are described as follows (Eq. 3-6), where q and c are the concentrations of component i=n solid and gas phase respectively. Equations (Eq.) 3-6 are as follows:

$$\frac{\partial {c}_{g , i}}{\partial t} = {D}_{ax} \frac{{\partial }^{2} {c}_{g , i}}{\partial {z}^{2}} - \frac{\partial ( {c}_{g , i} {u}_{g} )}{\partial z} - \frac{( 1 - ϵ ) {ρ}_{s}}{ϵ} \frac{\partial {q}_{i}}{\partial t} \qquad ( 3 )$$

$$\frac{\partial {q}_{i}}{\partial t} = {\sum }_{k = 1}^{3} \frac{\partial {q}_{k , i}}{\partial t} \qquad ( 4 )$$

$$\frac{\partial {q}_{k , i}}{\partial t} = {k}_{ov} ( {q}_{eq , k , i} - {q}_{k , i} ) \qquad ( 5 )$$

$${q}_{eq , i} = {\sum }_{k = 1}^{3} \frac{{q}_{sat , k , i} {b}_{ki} {c}_{g , i}}{{\sum }_{j = C {O}_{2} , {N}_{2} , {H}_{2} O} {b}_{k , j} {c}_{g , j}} \qquad ( 6 )$$

q_{eq} is the equilibrium isotherm capacity of I-th component and is represented by either single, dual or triple Langmuir isotherm as required for the sorbent and index k refers to the number of sites used in the Langmuir isotherm model. The overall mass transfer coefficient, kₒᵥ (Eq. 7) is modeled using linear driving force as where is the effective diffusivity, D_{eff} and rₚ is the radius of the sorbent pellet. The D_{eff} can be calculated using molecular simulation framework. Equation (Eg.) 7 is as follows:

kₒᵥ=15D_{eff}/rₚ²

The energy balances for the various phases and components of the contactor involved viz. gas phase (Eq. 8) , sorbent phase (Eq. 9), wall of the contactor shell (Eq. 10), and finally the external heat transfer fluid (Eq. 11) are all modeled individually as follows. Heat transfer parameters such as heat capacity of the sorbent (Cₚₛ) can come from molecular simulation framework,

$$\frac{\partial {T}_{g}}{\partial t} = \frac{1}{ϵ {ρ}_{g} {C}_{pq}} ( {λ}_{g} \frac{{\partial }^{2} {T}_{g}}{\partial {z}^{2}} - ϵ {ρ}_{g} {C}_{pg} {u}_{g} \frac{\partial {T}_{g}}{\partial z} + \frac{6 ( 1 - ϵ )}{2 {r}_{p} {h}_{gs} ( {T}_{s} - {T}_{g} )} + \frac{π {d}_{is} h ϵ}{V} {h}_{gws} ( {T}_{ws} - {T}_{g} ) + \frac{π {d}_{ot} {h}_{nT} ϵ}{V} {h}_{g} Wt ) \qquad ( 8 )$$

$$\frac{\partial {T}_{s}}{\partial t} = \frac{1}{( 1 - ϵ ) {ρ}_{s} {C}_{ps}} ( {λ}_{s} \frac{{\partial }^{2} {T}_{s}}{\partial {z}^{2}} - {( 1 - ϵ )}^{2} {ρ}_{s}^{2} {C}_{ps} {\sum }_{i = C {O}_{2} , {N}_{2} , {H}_{2} O} {\sum }_{k = 1}^{k = 3} \frac{\partial {q}_{k , i}}{\partial t} Δ {H}_{k , i} + \frac{6 ( 1 - ϵ )}{2 {τ}_{p}} {h}_{gs} ( {T}_{g} - {T}_{s} ) + \frac{π {d}_{is} h ( 1 - ϵ )}{{V}_{WS}} {h}_{sWS} ( {T}_{WS} - {T}_{S} ) ) \qquad ( 9 )$$

$$\frac{\partial {T}_{WS}}{\partial t} = \frac{1}{{ρ}_{WS} {C}_{WS}} ( {λ}_{WS} \frac{{\partial }^{2} {T}_{WS}}{\partial {z}^{2}} + \frac{π {d}_{is} h ϵ}{{V}_{WS}} {h}_{gWS} ( {T}_{g} - {T}_{WS} ) + \frac{π {d}_{is} h ( 1 - ϵ )}{{V}_{W s}} {h}_{sWS} ( {T}_{s} - {T}_{WS} ) + \frac{π {d}_{os} h}{{V}_{Ws}} {h}_{WSXE} ( {T}_{XE} - {T}_{WS} ) ) \qquad ( 10 )$$

$$\frac{\partial {T}_{XE}}{\partial t} = \frac{1}{{ρ}_{XE} {C}_{pXE}} ( {λ}_{XE} \frac{{\partial }^{2} {T}_{XE}}{\partial {z}^{2}} - {ρ}_{XE} {C}_{pXE} {u}_{XE} \frac{\partial {T}_{XE}}{\partial z} + \frac{π {d}_{os} h}{{V}_{XE}} {h}_{WSXE} ( {T}_{ws} - {T}_{XE} ) ) \qquad ( 11 )$$

The momentum balance is modeled using Ergun equation (Eq 12) as follows:

$$\frac{\partial P}{\partial z} = - \frac{1 \; 50 μ {( 1 - ϵ )}^{2} ϵ {u}_{g}}{{{ϵ}^{3} ( 2 {r}_{p} )}^{∧} 3} - \frac{1.75 ( 1 - ϵ ) {{ρ}_{g} ( {u}_{q} ϵ )}^{2}}{{ϵ}^{3} ( 2 {r}_{p} )} \qquad ( 12 )$$

The total pressure at every spatial and temporal point is determined using ideal gas law.

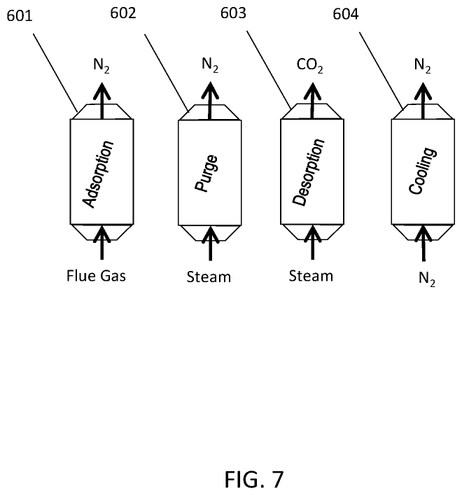

The cycle is configured to consists of four steps viz, adsorption 601, purge 602, desorption 603 and cooling 604. The schematic representation of the TSA cycle steps are shown in FIG. 7.

All spatial variables are discretized in gPROMS® and are solved using the DASOLV solver of gPROMS. Each material (selected under molecular simulation framework) is run until cyclic steady state is reached, whose criteria is based on monitoring whether the purity and recovery values of all components involved in addition to the exit temperature reaches a steady value within an appropriate tolerance level. The gPROMS® model is invoked from a Python wrapper, which provides the input files for each material, determining the cycle operating conditions and post-process the output to yield the figures of merit, such as illustrated in block 15.

The process based ranking and screening of the materials is done at two separate levels as described in FIG. 6. For the first level of screening, the feed gas velocity and the desorption temperature are set at some particular values and the step times are determined by performing a heuristic based optimization at block 13. The process model described using Equations (Eq.) 1 and 2 is run for the entire set of materials at the determined step times and other operating conditions and the figures of merit namely purity, recovery, gravimetric productivity and energy consumption are computed and extracted as the process model output for each material. Purity, recovery, specific energy consumption and volumetric productivity are defined as:

$${CO}_{2} purity [ \% ] = cumulative mole fraction of {CO}_{2} in desorption part of the cycle excluding steam$$

$${CO}_{2} recovery [ \% ] = \frac{cumulative {CO}_{2} recovered in desorption step}{total {CO}_{2} feed to the column during adsorption step}$$

$$Specific energy consumption [ MJ per Kg of {CO}_{2} ] = \frac{Energy of Inlet Streams - Energy of Outlet Streams + Energy of Steamin purge \& desorption}{total {CO}_{2} recovered over one cycle}$$

$$Volumetric productivity [ kg of {CO}_{2} per day per L of sorbent ] = \frac{cumulative {CO}_{2} recovered in desorption step}{Effective column volume for entire cycle \times ( 1 - bulk voidage )}$$

Referring back to FIG. 5, following the above described process optimizations at block 13 and 14, figures of merit from the process modeling framework are obtained and then used in combination with the molecular level properties (also figures of merit) from the molecular modeling framework to obtain material synthesis priority at block 16. This provides a ranking of sorbent materials for post combustion carbon capture, which is reported by the Combined microscopic performance and macroscopic process feasibility generator 28.

In some embodiments, in view of the outputs from the molecular level screening workflow described in FIG. 3 (molecular level screening workflow 26) and the process modeling framework described in FIG. 5 (process modeling workflow 27), a pareto-optimality condition may be selected to rank order materials based on microscopic as well as macroscopic figures of merits. “Pareto optimality” is the state at which resources in a given system are optimized in a way that one dimension cannot improve without a second worsening. Out of 4 process based figures of merits described above, there is a trade-off between all of them. For example there is a trade-off between CO₂ purity and CO₂ recovery. However the trade-off is such that it takes the form of inverted hockey stick shape. A knee point of such curve represents pareto optimality in two dimensions. Similar pareto optimality condition in multi-dimension space can be explored through optimization.

In some embodiments, the methods and systems described with reference to FIGS. 1-7 provide a gas capture and separation materials synthesis priority rank ordering system and method that includes an integrated Molecular modeling framework and Process Modeling framework to calculate process-level figures of merits based on microscopic properties of materials. In some embodiments, a molecular modeling framework is provided to calculate microscopic properties/figures-of-merit including, but not limited to, molecular transport properties (diffusivity, loading and mass transfer) and heat transport properties (heat capacity and heat transfer) at a range of pressures and temperatures. The methods and systems of the present disclosure can reduce discrete properties into a functional form to predict microscopic properties including, but not limited to, multicomponent loading at given temperatures and pressure, multicomponent diffusivities at given temperatures and pressures, heat capacity and heat transfer coefficient. In some embodiments, the systems and methods include a process modeling framework for quantifying separation process performance using microscopic molecular and heat transport properties.

In some embodiments, the methods and systems described with reference to FIGS. 1-7 provide a gas capture and separation materials synthesis priority rank ordering system and method that can determine an optimum operating condition for separation process by maximizing purity, recovery and throughput and minimizing specific energy use. The optimization can be carried out at two levels using simple process optimization to compared materials performances and the down selecting materials to run detailed process optimization for final rank ordering for synthesis priority. In some embodiments, ranking of materials may further include selecting a pareto-optimality condition to rank order materials based on microscopic as well as macroscopic figures of merits.

In another aspect, a method for carbon capture is described, as depicted in FIG. 1. The method can include characterizing sorbent materials with a molecular model workflow 26 that generates microscopic figures of merit for materials by microscopic properties; and evaluating the materials from the molecular model workflow 26 with a process model workflow 27 that generates macroscopic figures of merit for process steps of a carbon recovery process. The method can further include ranking the materials for applicability as a sorbent material using a combined microscopic performance and macroscopic process feasibility generator that ranks the materials according to the microscopic figures of merit for materials and the macroscopic figures of merit for the process steps. The highest ranked materials can be selected from the ranking for integration as sorbent materials into at least one of a desorber 62 and an adsorber 61 of a carbon capture process employing pressure and/or temperature swing adsorption cycles (PSA/TSA) 60, such as the carbon recapture process for a natural gas power plant 55, as depicted in FIG. 1. The method can then perform carbon recapture using the at least one of the desorber 61 and adsorber 62 with the sorbent materials.

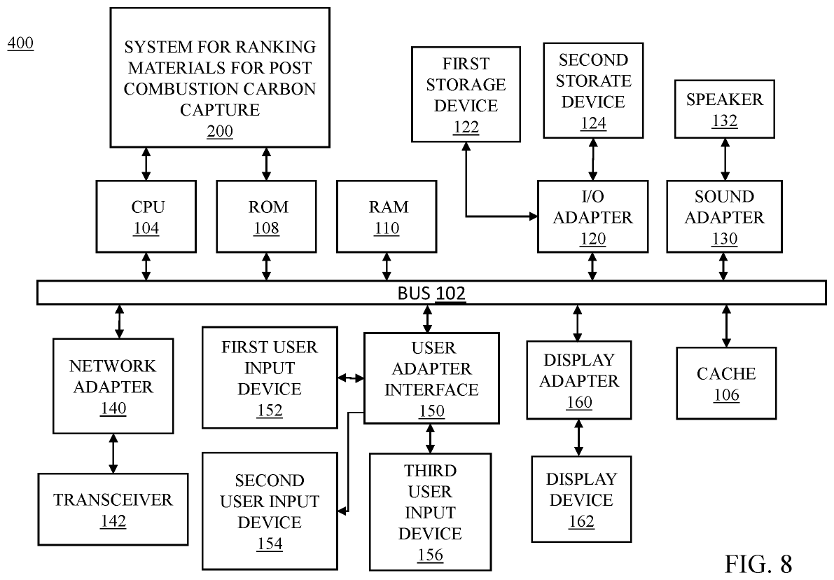

FIG. 8 further illustrates a processing system 400 that can include the system 200 for ranking materials described with reference to FIGS. 1-7. The exemplary processing system 400 to which the present invention may be applied is shown in accordance with one embodiment. The processing system 400 includes at least one processor (CPU) 104 operatively coupled to other components via a system bus 102. The system bus 102 may be in communication with the system for ranking materials for post combustion carbon capture 200. A cache 106, a Read Only Memory (ROM) 108, a Random Access Memory (RAM) 110, an input/output (I/O) adapter 120, a sound adapter 130, a network adapter 140, a user interface adapter 150, and a display adapter 160, are operatively coupled to the system bus 102. As illustrated, the system 100 that provides for provenance based identification of policy deviations in cloud environments can be integrated into the processing system 400 by connection to the system bus 102.

A first storage device 122 and a second storage device 124 are operatively coupled to system bus 102 by the I/O adapter 120. The storage devices 122 and 124 can be any of a disk storage device (e.g., a magnetic or optical disk storage device), a solid state magnetic device, and so forth. The storage devices 122 and 124 can be the same type of storage device or different types of storage devices.

A speaker 132 is operatively coupled to system bus 102 by the sound adapter 130. A transceiver 142 is operatively coupled to system bus 102 by network adapter 140. A display device 162 is operatively coupled to system bus 102 by display adapter 160.

A first user input device 152, a second user input device 154, and a third user input device 156 are operatively coupled to system bus 102 by user interface adapter 150. The user input devices 152, 154, and 156 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 152, 154, and 156 can be the same type of user input device or different types of user input devices. The user input devices 152, 154, and 156 are used to input and output information to and from system 400.

Of course, the processing system 400 may also include other elements (not shown), as readily contemplated by one of skill in the art, as well as omit certain elements. For example, various other input devices and/or output devices can be included in processing system 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 400 are readily contemplated by one of ordinary skill in the art given the teachings of the present invention provided herein.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), FPGAs, and/or PLAs.

These and other variations of a hardware processor subsystem are also contemplated in accordance with embodiments of the present invention.

The present invention may be a system, a method, and/or a computer program product at any possible technical detail level of integration. For example, in some embodiments, a computer program product is provided for ranking materials for post combustion carbon capture. The computer program product may include a computer readable storage medium. The computer readable storage medium may have computer readable program code embodied therewith, the program instructions executable by a processor to cause the processor to characterize, using the processor, sorbent materials with a molecular model workflow 26 that generates microscopic figures of merit for materials by microscopic properties; and evaluate, using the processor, the materials from the molecular model workflow with a process model workflow 27 that generates macroscopic figures of merit for process steps of a carbon recovery process. The computer readable storage medium also includes instructions that can rank (with the combined microscopic performance and macroscopic process feasibility generator 29), using the processor, the materials for applicability as a sorbent material using a combined microscopic performance and macroscopic process feasibility generator that ranks the materials according to the microscopic figures of merit for materials and the macroscopic figures of merit for the process steps.

The computer program product may include a computer readable storage medium (or media) having computer readable program instructions thereon for causing a processor to carry out aspects of the present invention. The computer program produce may also be non-transitory.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing.

A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

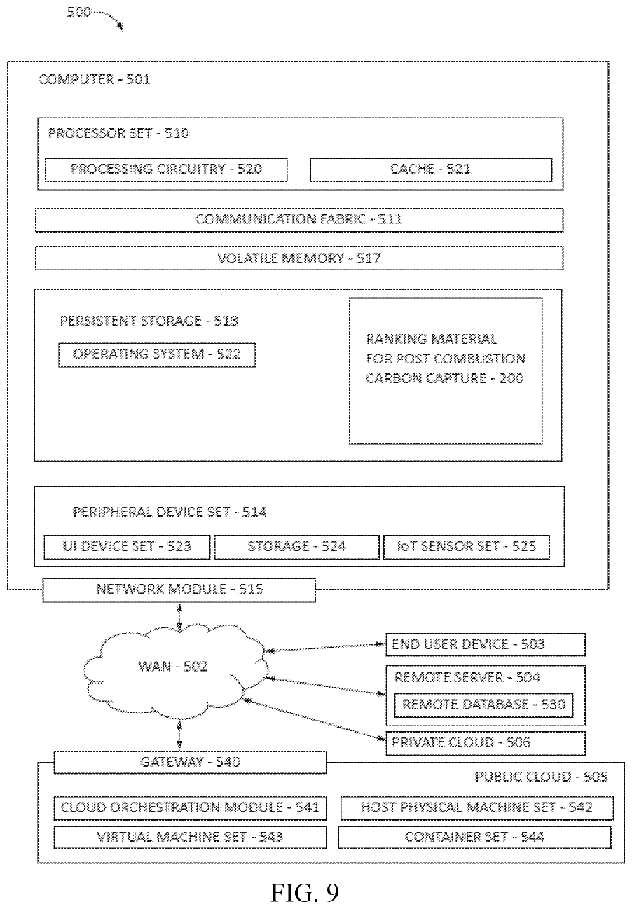

Referring to FIG. 9, the computing environment 500 contains an example of an environment for the execution of at least some of the computer code involved in performing the inventive methods, such as the method for ranking materials for post combustion carbon capture 200. In addition to block 200, computing environment 500 includes, for example, computer 501, wide area network (WAN) 502, end user device (EUD) 503, remote server 504, public cloud 505, and private cloud 506. In this embodiment, computer 501 includes processor set 510 (including processing circuitry 520 and cache 521), communication fabric 511, volatile memory 512, persistent storage 513 (including operating system 522 and block 200, as identified above), peripheral device set 514 (including user interface (UI), device set 523, storage 524, and Internet of Things (IoT) sensor set 525), and network module 515. Remote server 504 includes remote database 530. Public cloud 505 includes gateway 540, cloud orchestration module 541, host physical machine set 542, virtual machine set 543, and container set 544.

COMPUTER 501 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 530. As is well understood in the art of computer technology, and depending upon the technology, performance of a computer-implemented method may be distributed among multiple computers and/or between multiple locations. On the other hand, in this presentation of computing environment 500, detailed discussion is focused on a single computer, specifically computer 501, to keep the presentation as simple as possible.

Computer 501 may be located in a cloud, even though it is not shown in a cloud in FIG. 9. On the other hand, computer 501 is not required to be in a cloud except to any extent as may be affirmatively indicated.

PROCESSOR SET 510 includes one, or more, computer processors of any type now known or to be developed in the future. Processing circuitry 520 may be distributed over multiple packages, for example, multiple, coordinated integrated circuit chips. Processing circuitry 520 may implement multiple processor threads and/or multiple processor cores. Cache 521 is memory that is located in the processor chip package(s) and is typically used for data or code that should be available for rapid access by the threads or cores running on processor set 510. Cache memories are typically organized into multiple levels depending upon relative proximity to the processing circuitry. Alternatively, some, or all, of the cache for the processor set may be located “off chip.” In some computing environments, processor set 510 may be designed for working with qubits and performing quantum computing.

Computer readable program instructions are typically loaded onto computer 501 to cause a series of operational steps to be performed by processor set 510 of computer 501 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 521 and the other storage media discussed below. The program instructions, and associated data, are accessed by processor set 510 to control and direct performance of the inventive methods. In computing environment 500, at least some of the instructions for performing the inventive methods may be stored in block 200 in persistent storage 513.

COMMUNICATION FABRIC 511 is the signal conduction paths that allow the various components of computer 501 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 512 is any type of volatile memory now known or to be developed in the future. Examples include dynamic type random access memory (RAM) or static type RAM. Typically, the volatile memory is characterized by random access, but this is not required unless affirmatively indicated. In computer 501, the volatile memory 512 is located in a single package and is internal to computer 501, but, alternatively or additionally, the volatile memory may be distributed over multiple packages and/or located externally with respect to computer 501.

PERSISTENT STORAGE 513 is any form of non-volatile storage for computers that is now known or to be developed in the future. The non-volatility of this storage means that the stored data is maintained regardless of whether power is being supplied to computer 501 and/or directly to persistent storage 513. Persistent storage 513 may be a read only memory (ROM), but typically at least a portion of the persistent storage allows writing of data, deletion of data and re-writing of data. Some familiar forms of persistent storage include magnetic disks and solid state storage devices. Operating system 522 may take several forms, such as various known proprietary operating systems or open source Portable Operating System Interface type operating systems that employ a kernel. The code included in block 200 typically includes at least some of the computer code involved in performing the inventive methods.

PERIPHERAL DEVICE SET 514 includes the set of peripheral devices of computer 501. Data communication connections between the peripheral devices and the other components of computer 501 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 523 may include components such as a display screen, speaker, microphone, wearable devices (such as goggles and smart watches), keyboard, mouse, printer, touchpad, game controllers, and haptic devices. Storage 524 is external storage, such as an external hard drive, or insertable storage, such as an SD card. Storage 524 may be persistent and/or volatile. In some embodiments, storage 524 may take the form of a quantum computing storage device for storing data in the form of qubits. In embodiments where computer 101 is required to have a large amount of storage (for example, where computer 501 locally stores and manages a large database) then this storage may be provided by peripheral storage devices designed for storing very large amounts of data, such as a storage area network (SAN) that is shared by multiple, geographically distributed computers. IoT sensor set 525 is made up of sensors that can be used in Internet of Things applications. For example, one sensor may be a thermometer and another sensor may be a motion detector.

NETWORK MODULE 515 is the collection of computer software, hardware, and firmware that allows computer 101 to communicate with other computers through WAN 102. Network module 515 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 515 are performed on the same physical hardware device. In other embodiments (for example, embodiments that utilize software-defined networking (SDN)), the control functions and the forwarding functions of network module 515 are performed on physically separate devices, such that the control functions manage several different network hardware devices. Computer readable program instructions for performing the inventive methods can typically be downloaded to computer 501 from an external computer or external storage device through a network adapter card or network interface included in network module 515. WAN 502 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 503 is any computer system that is used and controlled by an end user (for example, a customer of an enterprise that operates computer 501), and may take any of the forms discussed above in connection with computer 501. EUD 503 typically receives helpful and useful data from the operations of computer 501. For example, in a hypothetical case where computer 501 is designed to provide a recommendation to an end user, this recommendation would typically be communicated from network module 515 of computer 501 through WAN 502 to EUD 503. In this way, EUD 503 can display, or otherwise present, the recommendation to an end user. In some embodiments,

EUD 503 may be a client device, such as thin client, heavy client, mainframe computer, desktop computer and so on.

REMOTE SERVER 504 is any computer system that serves at least some data and/or functionality to computer 501. Remote server 504 may be controlled and used by the same entity that operates computer 501. Remote server 504 represents the machine(s) that collect and store helpful and useful data for use by other computers, such as computer 501. For example, in a hypothetical case where computer 501 is designed and programmed to provide a recommendation based on historical data, then this historical data may be provided to computer 501 from remote database 530 of remote server 504.

PUBLIC CLOUD 505 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 505 is performed by the computer hardware and/or software of cloud orchestration module 541. The computing resources provided by public cloud 505 are typically implemented by virtual computing environments that run on various computers making up the computers of host physical machine set 542, which is the universe of physical computers in and/or available to public cloud 505. The virtual computing environments (VCEs) typically take the form of virtual machines from virtual machine set 543 and/or containers from container set 544. It is understood that these VCEs may be stored as images and may be transferred among and between the various physical machine hosts, either as images or after instantiation of the VCE. Cloud orchestration module 541 manages the transfer and storage of images, deploys new instantiations of VCEs and manages active instantiations of VCE deployments. Gateway 540 is the collection of computer software, hardware, and firmware that allows public cloud 505 to communicate through WAN 502.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 506 is similar to public cloud 505, except that the computing resources are only available for use by a single enterprise. While private cloud 506 is depicted as being in communication with WAN 502, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 505 and private cloud 506 are both part of a larger hybrid cloud.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

Having described preferred embodiments of a system and method for ranking materials for post combustion carbon capture (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

1. A computer implemented method for ranking materials for post combustion carbon capture comprising:
characterizing sorbent materials with a molecular model workflow that generates microscopic figures of merit for materials by microscopic properties;
evaluating the materials from the molecular model workflow with a process model workflow that generates macroscopic figures of merit for process steps of a carbon recovery process; and
ranking the materials for applicability as a sorbent material using a combined microscopic performance and macroscopic process feasibility generator that ranks the materials according to the microscopic figures of merit for materials and the macroscopic figures of merit for the process steps.

2. The computer implemented method for ranking materials for post combustion carbon capture of claim 1, wherein the microscopic properties are selected from the group consisting of loading, heat capacity, heat transfer and combinations thereof.

3. The computer implemented method for ranking materials for post combustion carbon capture, of claim 1, wherein the microscopic figures of merit include data from an adsorption isotherm for a particular sorbent material.

4. The computer implemented method for ranking materials for post combustion carbon capture of claim 1, wherein the macroscopic figures of merit for the carbon recovery process employing at least one of temperature swing adsorption cycle processes and pressure swing adsorption cycle processes are selected from the group consisting of recovery, purity, production, specific energy and combinations thereof for the product stream of interest.

5. The computer implemented method for ranking materials for post combustion carbon capture of claim 1 further comprising:
selecting highest ranked materials from the ranking for integration as sorbent materials into at least one of a desorber and an adsorber of a carbon capture process employing at least one of pressure swing adsorption cycles and temperature swing adsorption cycles; and
performing carbon recapture using the at least one of the desorber and adsorber with the sorbent materials.

6. The computer implemented method for ranking materials for post combustion carbon capture of claim 1, wherein the sorbent material is selected from the group consisting of zeolites, metal organic frameworks (MOF), zeolitic imidazolate frameworks (ZIF), porous polymer networks (PPN) and combinations thereof.

7. The computer implemented method for ranking materials for post combustion carbon capture of claim 1, wherein a combined microscopic performance and macroscopic process feasibility generator comprises of a multi-step and multi-criteria optimizer that orders the materials using combined trade-off metrics considering different dimensions of performance enhancement.

8. A system for preventing propagation of pathogens comprising:
a hardware processor; and
a memory that stores a computer program product, which, when executed by the hardware processor, causes the hardware processor to:
characterize sorbent materials with a molecular model workflow that generates microscopic figures of merit for materials by microscopic properties;
evaluate the materials from the molecular model workflow with a process model workflow that generates macroscopic figures of merit for process steps of a carbon recovery process; and
rank the materials for applicability as a sorbent material using a combined microscopic performance and macroscopic process feasibility generator that ranks the materials according to the microscopic figures of merit for materials and the macroscopic figures of merit for the process steps.

9. The system for ranking materials for post combustion carbon capture of claim 8, wherein the microscopic properties are selected from the group consisting of loading, heat capacity, heat transfer and combinations thereof.

10. The system for ranking materials for post combustion carbon capture of claim 8, wherein the microscopic figures of merit include data from an adsorption isotherm for a particular sorbent material.

11. The system for ranking materials for post combustion carbon capture of claim 8, wherein the macroscopic figures of merit for the carbon recovery process employing at least one of temperature swing adsorption cycle processes and pressure swing adsorption cycle processes are selected from the group consisting of recovery, purity, production, specific energy and combinations thereof for the product stream of interest.

12. The system for ranking materials for post combustion carbon capture of claim 8 further comprising:
select highest ranked materials from the ranking for integration as sorbent materials into at least one of a desorber and an adsorber of a carbon capture process employing at least one of a temperature swing adsorption cycle and pressure swing adsorption cycle; and
perform carbon recapture using the at least one of the desorber and adsorber with the sorbent materials.

13. System for ranking materials for post combustion carbon capture of claim 8, wherein a combined microscopic performance and macroscopic process feasibility generator comprises of a multi-step and multi-criteria optimizer that orders the materials using combined trade-off metrics considering different dimensions of performance enhancement.

14. A computer program product for ranking materials for post combustion carbon capture comprising a computer readable storage medium having computer readable program code embodied therewith, the program instructions executable by a processor to cause the processor to:
characterize, using the processor, sorbent materials with a molecular model workflow that generates microscopic figures of merit for materials by microscopic properties;
evaluate, using the processor, the materials from the molecular model workflow with a process model workflow that generates macroscopic figures of merit for process steps of a carbon recovery process; and
rank, using the processor, the materials for applicability as a sorbent material using a combined microscopic performance and macroscopic process feasibility generator that ranks the materials according to the microscopic figures of merit for materials and the macroscopic figures of merit for the process steps.

15. The computer program product of claim 14, wherein the microscopic properties are selected from the group consisting of loading, heat capacity, heat transfer and combinations thereof.

16. The computer program product of claim 14, wherein the microscopic figures of merit include data from an adsorption isotherm for a particular sorbent material.

17. The computer program product of claim 14, wherein the macroscopic figures of merit for the carbon recovery process employing at least one of temperature swing adsorption cycle processes and pressure swing adsorption cycle processes are selected from the group consisting of recovery, purity, production, specific energy and combinations thereof for the product stream of interest.

18. The computer program product of claim 14 further comprising:
select, using the processor, highest ranked materials from the ranking for integration as sorbent materials into at least one of a desorber and an adsorber of a carbon capture process employing at least one of a temperature swing adsorption cycle and pressure swing adsorption cycle; and
perform, using the processor, carbon recapture using the at least one of the desorber and adsorber with the sorbent materials.

19. A method for separation process implementation comprising:
characterizing sorbent materials with a molecular model workflow that generates microscopic figures of merit for materials by microscopic properties;
evaluating the materials from the molecular model workflow with a process model workflow that generates macroscopic figures of merit for separation process steps of interest;
ranking the materials for applicability as a sorbent material using a combined microscopic performance and macroscopic process feasibility generator that ranks the materials according to the microscopic figures of merit for materials and the macroscopic figures of merit for the process steps;
selecting highest ranked materials from the ranking for integration as sorbent materials into at least one of a desorber and an adsorber of a separation process employing at least one of a pressure swing adsorption cycle and a temperature swing adsorption cycle; and
performing separation using the at least one of the desorber and adsorber with the sorbent materials.

20. The method of claim 19, wherein the microscopic properties are selected from the group consisting of loading, heat capacity, heat transfer and combinations thereof.

21. The method of claim 19, wherein the microscopic figures of merit include data from an adsorption isotherm for a particular sorbent material.

22. The method of claim 19, wherein the macroscopic figures of merit for the separation process employing at least one of temperature swing adsorption cycle processes and pressure swing adsorption cycle processes are selected from the group consisting of recovery, purity, production, specific energy and combinations thereof for the product stream of interest.

23. The method of claim 19, wherein the sorbent material is selected from the group consisting of zeolites, metal organic frameworks (MOF), zeolitic imidazolate frameworks (ZIF), porous polymer networks (PPN) and combinations thereof.

24. The method of claim 19, wherein the separation process is selected from the group consisting of carbon recovery, carbon capture, air separation, natural gas separation, hydrogen purification, ammonia separation, N₂ purification, O₂ purification, H₂O removal, bio gas separation and combinations thereof.

25. The computer implemented method for ranking materials for post combustion carbon capture comprising:
characterizing sorbent materials of metal organic frameworks with a molecular model workflow that generates microscopic figures of merit for materials by microscopic properties;
evaluating the materials from the molecular model workflow with a process model workflow that generates macroscopic figures of merit for process steps of a carbon recovery process; and
ranking the materials for applicability as a sorbent material using a combined microscopic performance and macroscopic process feasibility generator that ranks the materials according to the microscopic figures of merit for materials and the macroscopic figures of merit for the process steps.
